(19) United States
(12) Patent Application Publication
(10) Pub. No.: US 2007/0007368 A1
(43) Pub. Date: Jan. 11, 2007

(54) Air cooling device

(76) Inventor: Dodier Maxime, Montreal (CA)

Correspondence Address: INVENTARIUM, Suite 1607, 4050 Rosemont Blvd., Montreal, QC, H1X 1M4, CA

(21) Appl. No.: 11/482,584

(22) Filed: Jul. 6, 2006

Related U.S. Application Data
(60) Provisional application No. 60/696,642, filed on Jul. 6, 2005.

(51) Int. Cl. A24F 25/00; B05B 15/00; A61L 9/04

(52) U.S. Cl. 239/542; 239/34; 239/547

(57) ABSTRACT

An air cooling device works by first cooling a volume of liquid down to a temperature that can be below freezing. That volume of liquid can remain cold for a long period of time, requiring very little work on the part of the compressor, much like a kitchen refrigerator. Even when in use for cooling, besides the fan which is on all the time during use, the compressor is only required for short periods of time to maintain the cold temperaure of the liquid.

This application claims priority based on provisional patent application 60/696,642 filed Jul. 6, 2005

### BACKGROUND OF THE INVENTION

1. Field of the Invention

The invention relates generally to climate controlling devices but more particularly to an air cooling unit.

2. Background of the Invention

There are many types of air conditioning units as well as many types of fans used for cooling. Some inventions combine a fan with provision for ice or cold water so that air passing through the ice or cold water will be cooled.

Usually air conditioning unit need to be installed in a window or have some sort of air duct arrangement to take the air from the outside. This arrangement is of course elaborate in its installation There are also some types of portable air conditioning devices such as for example in Japanese patent JP2001132985.

There is still a need for a practical portable air conditioning unit which is quiet and unobtrusive.

### SUMMARY OF THE INVENTION

In view of the foregoing disadvantages inherent in the known devices now present in the prior art, the present invention, which will be described subsequently in greater detail, is to provide objects and advantages which are:

To provide for a small compact air cooling device which does not require to be installed in a window or require outside air.

To provide for a small compact air cooling device which is transporatble.

To provide for a small compact air cooling device which is quiet.

To provide for a small compact air cooling device which does not require the compressor to work as often as a standard air conditioning unit.

In order to do so, the invention works by first cooling a volume of liquid down to a temperature that can be below freezing. That volume of liquid can remain cold for a long period of time, requiring very little work on the part of the compressor, much like a kitchen refrigerator. Even when in use for cooling, besides the fan which is on all the time during use, the compressor is only required for short periods of time to maintain the cold temperaure of the liquid.

The device comprises a compressor, a condenser, refrigerant liquid circulating through refrigerant coils. The refrigerant coils are installed inside a liquid chamber which is filled with a liquid. A fan pulls ambient air through an air tunnel. The air tunnel has an input that is external to the body and outputs through the fan. The fan then blows the ambient air through pipes which pass through the liquid chamber. These pipes also have an input and an output with the input interfacing with the fan and the pipe output being at a location external to the body and distal from the air tunnel input. Insulating means insulate and seal the liquid chamber from within the body which keeps the liquid cold for a long period of time, thus reducing the frequency by which the compressor needs to work which reduces the amount of noise generated by the device.

The preferred cooling liquid in the liquid chamber is a brine solution so as to be cooled below the normal freezing temperature of water, but any other suitable liquid can be used.

In a preferred embodiment, the air tunnel input is diametrically opposite the pipe output but one can easily understand that there are a variety of possible arrangement all falling within the scope of the invention, given that the shape of the body can be of a variety of designs.

In order to increase the effective length of the pipes they can, in some embodiments, be given a sinusoidal shape.

Alternatively, the pipes can also be corkscrew in shape to further increase their effective lengths.

In a preferred embodiment, the air cooling device is supported by a stand or any such mechanical equivalent supporting structure.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE PREFERRED EMBODIMENT

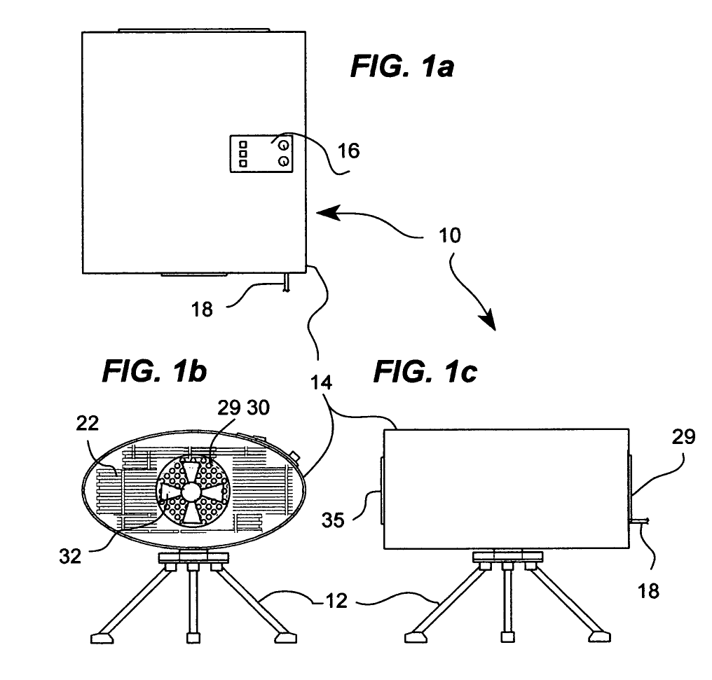

FIGS. 1a-c Top, rear and side views, respectively, of the invention.

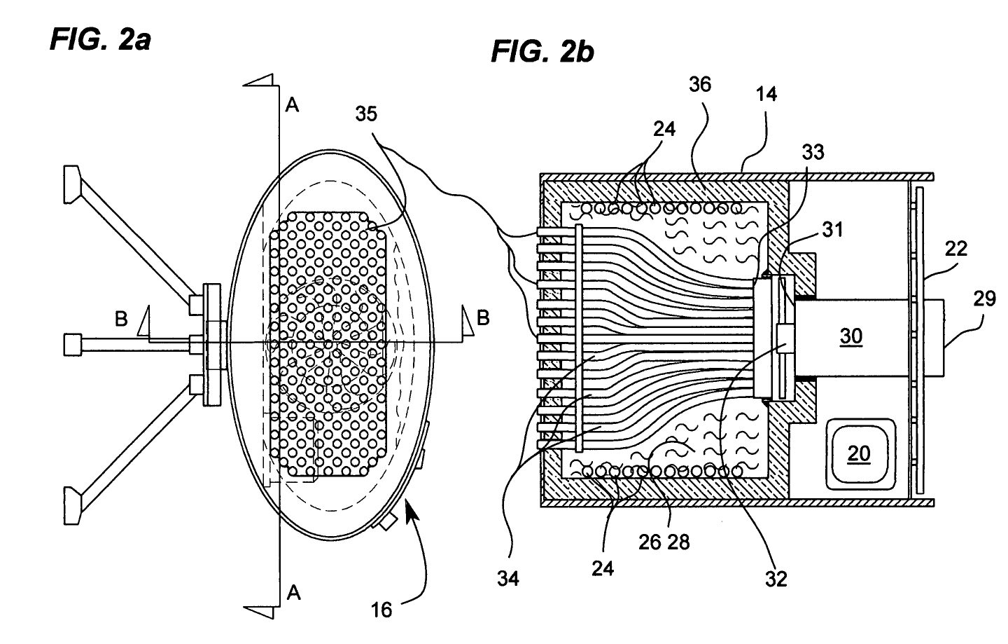

FIG. 2a Front view indicating cutaway line A-A.

FIG. 2b Top cutaway view along line A-A.

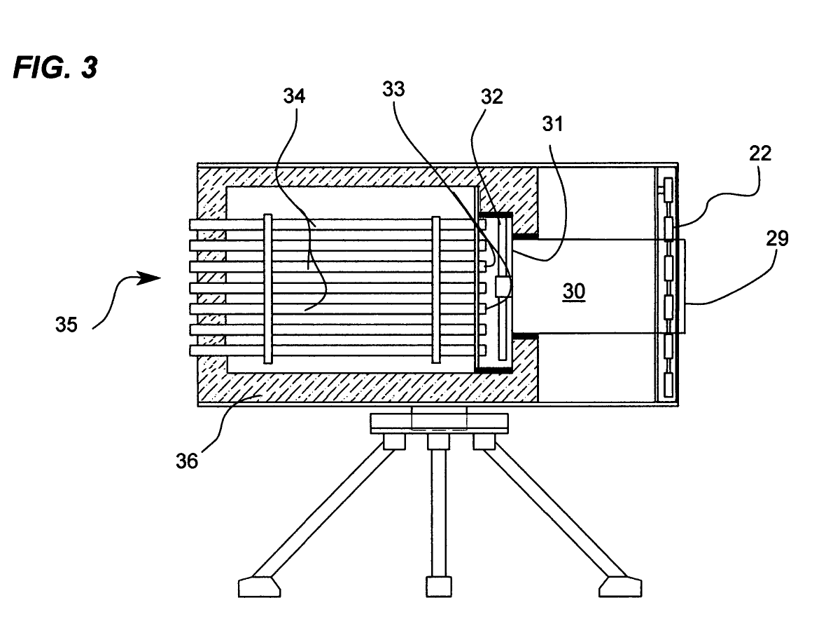

FIG. 3 Side cutaway view along line B-B.

### DETAILED DESCRIPTION OF THE PREFERRED EMBODIMENT

An air cooling device (10) has a stand (12), a body (14), a control panel (16), and a power cord (18). The body (14) contains a compressor (20) with a condenser (22) located on an external face of the body. Refrigerant coils (24) are installed inside a liquid chamber (26) filled with a liquid (28). The details of the compressor (20) and the refrigerant coils (24) need not be further discussed here since their workings are well known in the art.

An air tunnel (30), having a tunnel input (29) and a tunnel output (31), picks up ambient air from behind the air cooling device (10) and blows it through pipes (34) by way of a fan (32). The fan (32) is preferably of the computer cooling fan variety with a built-in motor so as to easily pass air without being too bulky. The pipes (34), which have a pipe input (33) and a pipe output (35), pass through the liquid chamber (26) which has liquid (28) surrounding each pipe (34) as they spread apart. Passing through the cold liquid (28) cools the air within the pipes (34).

The liquid chamber (26) is kept cold and hermetic by way of insulating means (36) surrounding it. Cooled air exits the pipes (34) at the pipe output (35). The control panel (16) controls such parameters as room temperature, fan (32) speed, etc. . . . An optional remote control (not shown) could also be a desirable option.

The body (14) can be pivotally connected to the stand (12) so that it can be oriented in any direction. Also, a motor (not shown) can pivot the body (14) so that the blown ambient air can be spread across a wider area, as is known of oscillating fans. This feature could also be controlled via the control panel (16) or remote control (not shown).

As seen in FIG. 3, the pipes (34) are rectilinear but, in order to increase their effective length, they could adopt a sinusoidal shape which would have the ambient air stay longer within the pipes (34) and thus cool even more. The same goes for the top view of FIG. 2b wherein the pipes (34), besides spreading apart, could also have a sinusoidal ondulation which, when taken together (side view, top view) gives the pipes (34) a three dimensional configuration akin to a corkscrew which would greatly increase their effective legth.

Although the air cooling device (10) preferably comes with its own stand (12) it is conceivable that it could work without it such as a desktop unit or that the stand (12) could be any of a variety made by third party manufacturers providing releasably attachable stands (12).

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An air cooling device for cooling a room having a body comprising:
a compressor, a condenser, refrigerant liquid circulating through refrigerant coils;
said refrigerant coils installed inside a liquid chamber and said liquid chamber being filled with a liquid;
a fan to pull ambient air through an air tunnel;
said air tunnel having a tunnel input and a tunnel output;
said input being external of said body;
said output being through said fan;
said fan blowing said ambient air through pipes;
said pipes passing through said liquid chamber;
said pipes having a pipe input and a pipe output;
said pipe input interfacing with said fan;
said pipe output being at a location external to said body and distal from said air tunnel input;
insulating means to insulate and seal said liquid chamber from within said body;

2. An air cooling device for cooling a room having a body as in claim 1 wherein:
said liquid being a brine solution.

3. An air cooling device for cooling a room having a body as in claim 1 wherein:
said air tunnel input being diametrically opposite said pipe output.

4. An air cooling device for cooling a room having a body as in claim 1 wherein:
said pipe being sinusoidal in shape.

5. An air cooling device for cooling a room having a body as in claim 1 wherein:
said pipe being corkscrew in shape.

6. An air cooling device for cooling a room having a body as in claim 1 wherein:
said body being supported by a stand.
